(19) United States
(12) Patent Application Publication
(10) Pub. No.: US 2026/0210878 A1
(43) Pub. Date: Jul. 23, 2026

(54) INSPECTION DEVICE FOR CAVITY-TYPE OBJECT

(71) Applicant: NUCTECH COMPANY LIMITED, Beijing (CN)

(72) Inventors: Jiawei DENG, Beijing (CN); Qinchan WANG, Beijing (CN); Jianhui WANG, Beijing (CN); Jinyu ZHANG, Beijing (CN)

(73) Assignee: NUCTECH COMPANY LIMITED, Beijing (CN)

(21) Appl. No.: 19/143,652

(22) PCT Filed: Dec. 25, 2023
(86) PCT No.: PCT/CN2023/141470

§ 371 Date: Jun. 26, 2025

(30) Foreign Application Priority Data
Dec. 28, 2022 (CN) 202211699230.2

(51) Int. Cl. G01N 23/046; G01N 33/12

(52) U.S. Cl. CPC G01N 23/046; G01N 33/12; G01N 2223/3308; G01N 2223/612

(57) ABSTRACT

Provided is an inspection device for a cavity-type object, including: a base; an inspection chamber rotatably disposed on the base, including: an inspection chamber body with an inspection channel having two open ends formed inside; an X-ray source and a radiation detector respectively disposed on opposite first and second side surfaces of the inspection channel; a conveyor belt extending through the inspection channel between the X-ray source and the radiation detector and configured to suspend and transport an object to be inspected; a first driving device disposed on the base and configured to drive the inspection chamber to rotate relative to the base to change an angle between rays emitted by the X-ray source and the conveyor belt. When the angle is equal to 90°, a distance between the conveyor belt and the first side surface is less than a distance between the conveyor belt and the second side surface.

### CROSS REFERENCE TO RELATED APPLICATION(S)

This application is a Section 371 National Stage Application of International Application No. PCT/CN 2023/141470, filed on Dec. 25, 2023, which claims priority to Chinese Patent Application No. 202211699230.2 filed on Dec. 28, 2022, the contents of which are incorporated herein by reference in its entirety.

### TECHNICAL FIELD

At least one embodiment of the present disclosure relates to an inspection device, and in particular, to an inspection device for a cavity-type object and may eliminate an influence of front and back occlusions.

### BACKGROUND

In daily lives, the meat that people commonly consume is typically cut meat. Taking pork as an example, the pork may be graded into three grades according to muscle development and fat thickness of a carcass, as well as muscle tissue structures of different sites of the carcass, the pork may also be graded into three grads according to edible values and processing uses. The pig carcass can be cut into six types of meat according to sites, including front leg meat, pork chops, square meat, belly, hind leg meat, hock, etc.

Traditional meat cutting methods rely on manual labor with in collaboration with machinery, depending on workers'experience. Such method is low in efficiency and requires a high level of workers'experience. Nowadays, automated cutting apparatuses are adopted in the slaughtering industry, in which it is needed to photograph the meat before cutting to identify proportions of fat, lean meat and bones and locate a position of ribs for subsequent cutting operations.

In the related art, an object to be inspected, such as a live pig, a live sheep, or poultry, is typically imaged using rays perpendicular (orthogonal) to a travelling direction of the object, or using spiral imaging, so as to inspect an internal structure of the object. However, due to the presence of the abdominal cavity, an orthogonal imaging may suffer from front and back occlusions, resulting in an unclear rear image and bringing difficulties to automatic recognition. Although such issues may be overcome by using the CT technology, the CT technology has problems of high costs, large space requirements, a large number of moving components, complex structure, and low practicality, making it unsuitable for promotion.

### SUMMARY

Embodiments of the present disclosure provide an inspection device for a cavity-type object.

According to an embodiment in an aspect of the present disclosure, an inspection device for a cavity-type object includes: a base; an inspection chamber rotatably disposed on the base, the inspection chamber includes: an inspection chamber body, an inspection channel with two open ends is formed inside the inspection chamber body; an X-ray source disposed on a first side surface of the inspection channel; and a radiation detector disposed on a second side surface of the inspection channel opposite to the first side surface; a conveyor belt extending through the inspection channel between the X-ray source and the radiation detector, the conveyor belt is configured to suspend and transport an object to be inspected; and a first driving device disposed on the base, the first driving device is configured to drive the inspection chamber to rotate relative to the base, so as to change an angle between rays emitted by the X-ray source and the conveyor belt. The conveyor belt is configured such that when the angle is equal to 90°, a distance between the conveyor belt and the first side surface is less than a distance between the conveyor belt and the second side surface.

According to some embodiments of the present disclosure, two X-ray sources are provided, and the two X-ray sources are horizontally arranged side by side and spaced apart on the first side surface of the inspection channel.

According to some embodiments of the present disclosure, the inspection device further includes: an image generation device communicatively connected to the radiation detector, the image generation device is configured to generate a three-dimensional image of the object to be inspected based on received ray signals.

According to some embodiments of the present disclosure, the object to be inspected is an animal carcass, and the three-dimensional image includes at least one of a skeletal image of the animal carcass, a muscle distribution image of the animal carcass, and a lean-fat contrast image of the animal carcass.

According to some embodiments of the present disclosure, the inspection chamber further includes a vertical movement assembly, and the vertical movement assembly includes: a guide rail disposed vertically on a side wall of the inspection channel; a sliding platform slidably connected to the guide rail, the two X-ray sources are disposed on the sliding platform; and a second driving device configured to drive the sliding platform to move in a vertical direction so as to change a ray emission height.

According to some embodiments of the present disclosure, the first driving device includes a servo motor.

According to some embodiments of the present disclosure, the second driving device includes any one of a linear motor, a pneumatic cylinder, and a hydraulic cylinder.

According to some embodiments of the present disclosure, an apparatus installation chamber is provided inside the inspection chamber, two through holes extending vertically are provided on a side wall of the apparatus installation chamber to connect the apparatus installation chamber and the inspection channel, and the two X-ray sources are configured to emit rays to the radiation detector through the two through holes respectively.

According to some embodiments of the present disclosure, an angle between an axis of the inspection channel and the conveyor belt ranges from −15° to 15°.

According to some embodiments of the present disclosure, two sliding platforms and two second driving devices are provided, the two X-ray sources are respectively disposed on the two sliding platforms, and ray emission heights of the two X-ray sources 202 may be the same or different.

### BRIEF DESCRIPTION OF THE DRAWINGS

FIG. 1 is a perspective view of an inspection device for a cavity-type object according to an exemplary embodiment of the present disclosure;

FIG. 2 is a schematic diagram of a working principle of the inspection device shown in FIG. 1;

FIG. 3 is a perspective view of an inspection device for a cavity-type object according to another exemplary embodiment of the present disclosure; and

FIG. 4 is a partial schematic structural diagram of the inspection device shown in FIG. 3.

### REFERENCE NUMERALS IN THE ACCOMPANYING DRAWINGS ARE AS FOLLOWS

1—base;
2—inspection chamber;
201—inspection channel;
202—X-ray source;
2021—first X-ray source;
2022—second X-ray source;
203—radiation detector;
204—rotation shaft;
3—conveyor belt;
4—first driving device;
5—object to be inspected;
6—sliding platform;
7—second driving device; and
8—through hole.

### DETAILED DESCRIPTION OF EMBODIMENTS

In order to make objectives, technical solutions and advantages of the present disclosure clearer, the present disclosure will be further described in detail below with reference to specific embodiments and the accompanying drawings.

However, it should be understood that these descriptions are only exemplary and are not intended to limit the scope of the present disclosure. In the following detailed descriptions, for the purpose of explanation, many specific details are set forth to provide a comprehensive understanding of the embodiments of the present disclosure. However, it is obvious that one or more embodiments may also be implemented without these specific details. Additionally, in the following descriptions, descriptions of well-known technologies are omitted to avoid unnecessarily confusing the concept of the present disclosure.

The terms used here are only for describing specific embodiments and are not intended to limit the present disclosure. The term “including” used here indicates the presence of features, steps and operations, but does not exclude the presence or addition of one or more other features.

When an expression similar to “at least one of A, B and C” is used, it should generally be interpreted as the meaning commonly understood by those skilled in the art (for example, “a system having at least one of A, B and C” should include but not be limited to a system having A alone, a system having B alone, a system having C alone, a system having A and B, a system having A and C, a system having B and C, and/or a system having A, B and C, etc.). When an expression similar to “at least one of A, B or C” is used, it should generally be interpreted as the meaning commonly understood by those skilled in the art (for example, “a system having at least one of A, B or C” should include but not be limited to a system having A alone, a system having B alone, a system having C alone, a system having A and B, a system having A and C, a system having B and C, and/or a system having A, B and C, etc.).

All terms (including technical and scientific terms) used here have the meanings commonly understood by those skilled in the art unless otherwise defined. It should be noted that the terms used here should be interpreted as having meanings consistent with the context of this specification and should not be interpreted in an idealized or overly rigid manner.

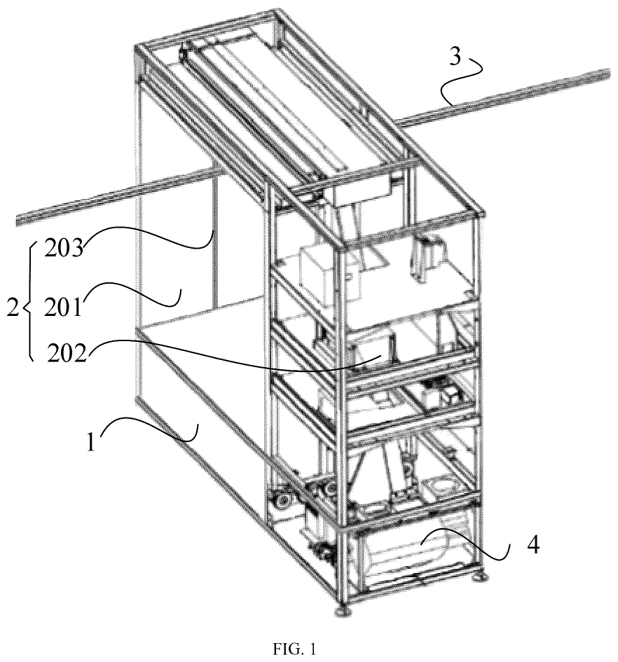

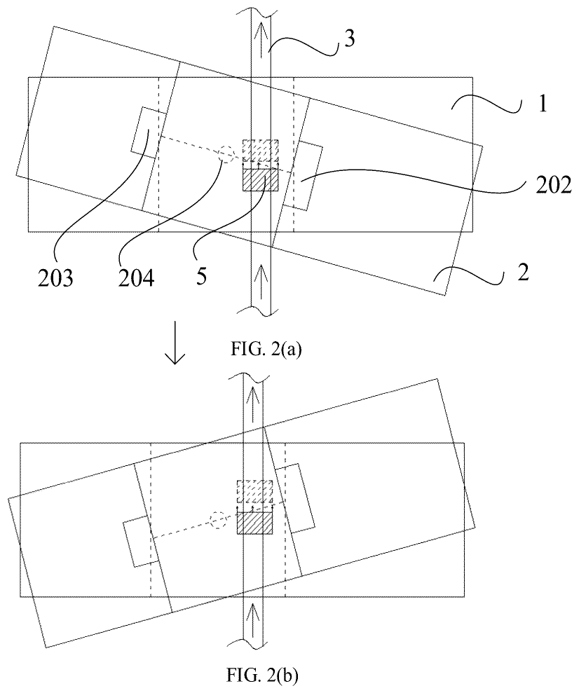

FIG. 1 is a perspective view of an inspection device for a cavity-type object according to an exemplary embodiment of the present disclosure. FIG. 2 is a schematic diagram of a working principle of the inspection device shown in FIG. 1.

According to an embodiment in an aspect of the present disclosure, an inspection device for a cavity-type object is provided, which may perform multi-angle imaging in a non-orthogonal manner when inspecting a cavity-type object, thereby effectively avoiding front and back occlusions that may cause an unclear rear image and providing more accurate and clear X-ray images.

As shown in FIG. 1 and FIG. 2, the inspection device for a cavity-type object according to an embodiment in an aspect of the present disclosure includes a base 1, an inspection chamber 2, a conveyor belt 3, and a first driving device 4. The inspection chamber 2 is rotatably disposed on the base 1. The inspection chamber includes: an inspection chamber body, where an inspection channel 201 with two open ends is formed inside the inspection chamber; an X-ray source 202 disposed on a first side surface of the inspection channel 201; and a radiation detector 203 disposed on a second side surface of the inspection channel 201 opposite to the first side surface. The conveyor belt 3 extends through the inspection channel 201 between the X-ray source 202 and the radiation detector 203, and the conveyor belt 3 is used to suspend and transport an object 5 to be inspected. The first driving device 4 is disposed on the base 1, and the first driving device 4 is configured to drive the inspection chamber 2 to rotate relative to the base 1 so as to change an angle between rays emitted by the X-ray source 202 and the conveyor belt 3. The conveyor belt 3 is configured such that when the angle is equal to 90°, a distance between the conveyor belt 3 and the first side surface is less than a distance between the conveyor belt 3 and the second side surface.

In this embodiment, by using the driving device to drive the inspection chamber 2 to rotate relative to the base 1 so as to change the angle between the rays emitted by the X-ray source 202 and the conveyor belt 3, so that multi-position and multi-angle imaging may be achieved during a movement of the object 5 to be inspected along with the conveyor belt 3, thereby avoiding an unclear rear image caused by front and back occlusions.

According to some embodiments of the present disclosure, the base 1 is a support structure of the device. A rotation shaft 204 with an axis extending in a vertical direction is disposed on the base 1. The inspection chamber 2 is connected to the rotation shaft 204, and the inspection chamber 2 may rotate about the axis of the rotation shaft 204.

According to some embodiments of the present disclosure, the first driving device 4 includes a servo motor, and the first driving device 4 is in transmission connection with the inspection chamber 2. Optionally, a transmission mode includes chain transmission, gear transmission, belt transmission, etc.

According to some optional embodiments of the present disclosure, the inspection channel 201 is a rectangular channel, and the axis of the rotation shaft 204 coincides with a vertical central axis of the inspection channel 201. The conveyor belt 3 extends transversely through the inspection channel 201. Optionally, the conveyor belt 3 is located in an upper space of the inspection channel 201. As shown in FIG. 2, the conveyor belt 3 is offset from a transversal central line of the inspection channel 201. Specifically, when the first driving device 4 drives the inspection chamber 2 to rotate to a position where the conveyor belt 3 is perpendicular to a direction of rays, that is, when the transversal central axis of the inspection channel 201 is parallel to the conveyor belt 3, a distance between the conveyor belt 3 and the first side surface is less than a distance between the conveyor belt 3 and the second side surface, that is, the object 5 to be inspected that is transported on the conveyor belt 3 is closer to the X-ray source 202 than to the radiation detector 203.

A working principle and a working process of the inspection device of the present disclosure will be described below with reference to FIG. 2 and a specific embodiment. It should be understood that this specific embodiment is merely provided for better understanding of the solution by those skilled in the art, and should not be interpreted as limiting the scope of protection of the present disclosure. In addition, FIG. 2 is merely a schematic diagram of the inspection device of the present disclosure, and sizes in FIG. 2 are not drawn proportionally to actual scale. For clarity, some local structures have been enlarged. Therefore, the relative sizes and positional relationships of components should not be regarded as limitations to the inspection device of the present disclosure.

As shown in FIG. 2(a), when the object 5 to be inspected enters the inspection channel 201, the first driving device 4 drives the inspection chamber 2 to rotate to a position shown in FIG. 2(a). During a process of the object 5 to be inspected moving from a position shown in the drawing to a position indicated by a dashed box, the rays emitted by the X-ray source 202 penetrate the object 5 to be inspected and are absorbed by the radiation detector 203, so that the object 5 to be inspected undergoes a first imaging process.

When the object 5 to be inspected moves to the position indicated by the dashed box in FIG. 2(a), the first driving device 4 drives the inspection chamber 2 to rotate counterclockwise to a position shown in FIG. 2(b). A relative positional relationship between the object 5 to be inspected, the X-ray source 202, and the radiation detector 203 is as shown in FIG. 2(b). The forwarding of the object 5 requires passing through the X-rays emitted by the X-ray source 202 for another time. When the object 5 to be inspected moves from the position shown in FIG. 2(b) to a position indicated by a dashed box, the object 5 to be inspected undergoes a second imaging process.

Based on the above working process, the object 5 to be inspected may undergo two imaging processes in two sections respectively when being inspected inside the inspection channel 201. In the two sections, the rays irradiate the object 5 to be inspected at different angles, and the two imaging processes are both performed in a non-orthogonal mode, which maximally avoids defects in the related art that a rear image is unclear due to front and back occlusions.

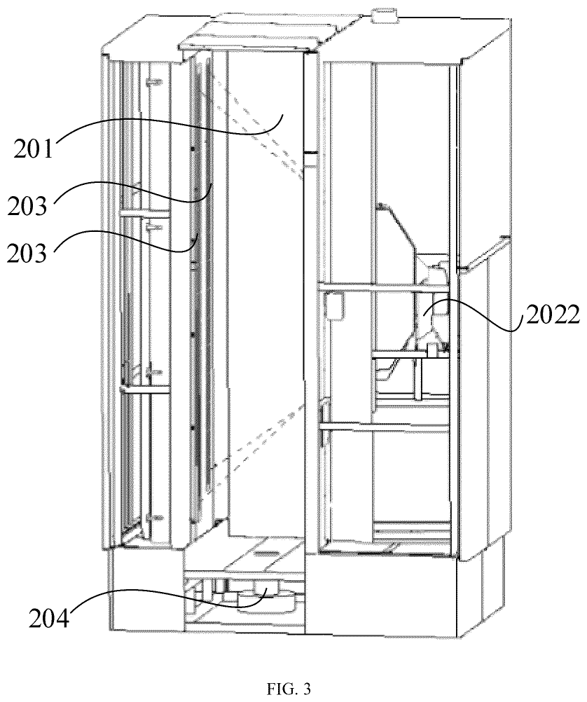

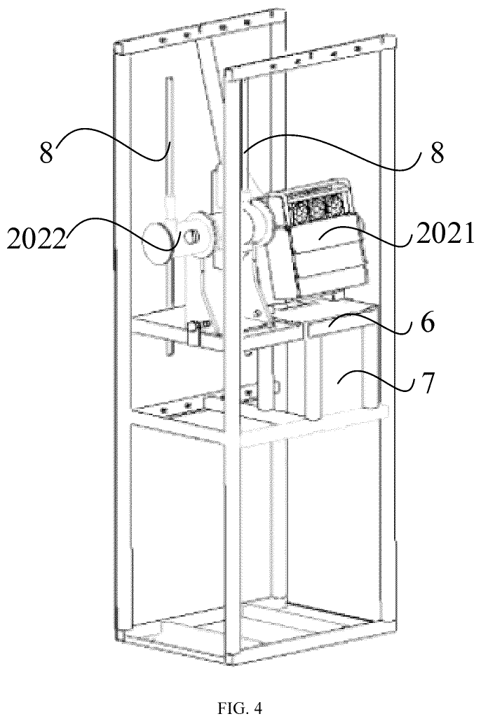

FIG. 3 is a perspective view of an inspection device for a cavity-type object according to another exemplary embodiment of the present disclosure. FIG. 4 is a partial schematic structural diagram of the inspection device shown in FIG. 3.

According to some embodiments of the present disclosure, as shown in FIG. 3 and FIG. 4, two X-ray sources 202 are provided, and the two X-ray sources 202 are horizontally arranged side by side and spaced apart on the first side surface of the inspection channel 201.

In this embodiment, a first X-ray source 2021 and a second X-ray source 2022 are arranged side by side, and the rays emitted by the first and second X-ray sources are received by the radiation detector 203 (array). Optionally, the first X-ray source 2021 and the second X-ray source 2022 may emit rays of different energy intensities. Based on different X-ray absorption capacities (radiodensities) of bones, lean meat, and fat, a structure and a composition of the object 5 to be inspected may be imaged better for better differentiation.

According to some embodiments of the present disclosure, the inspection device further includes an image generation device, which is communicatively connected to the radiation detector 203. The image generation device is configured to generate a three-dimensional image of the object to be inspected based on received ray signals, and the three-dimensional image is applicable for guiding subsequent cutting operations.

According to some embodiments of the present disclosure, the object 5 to be inspected is an animal carcass, and the three-dimensional image includes at least one of a skeletal image, a muscle distribution image, and a lean-fat contrast image of the animal carcass.

According to some embodiments of the present disclosure, the inspection chamber further includes a vertical movement assembly, including a guide rail (not shown), a sliding platform 6, and a second driving device 7. The guide rail is disposed vertically on a side wall of the inspection channel 201. The sliding platform 6 is slidably connected to the guide rail, and the two X-ray sources 202 are disposed on the sliding platform 6. The second driving device 7 is configured to drive the sliding platform 6 to move in the vertical direction so as to change a ray emission height.

In this embodiment, when the object 5 to be inspected includes carcasses of different types of animals, such as pigs, cattle, sheep, chickens, ducks, geese, etc., or of the same type but different batches with different average body sizes, it is needed to adjust a height of the X-ray source 202 for more accurate imaging of the object 5 to be inspected.

According to some embodiments of the present disclosure, the second driving device 7 includes any one of a linear motor, a pneumatic cylinder, and a hydraulic cylinder.

According to some embodiments of the present disclosure, an apparatus installation chamber is provided inside the inspection chamber, and two through holes 8 extending vertically are provided on a side wall of the apparatus installation chamber to connect the apparatus installation chamber and the inspection channel 201. The two X-ray sources 202 emit rays to two radiation detectors 203 respectively through the two through holes 8.

According to some embodiments of the present disclosure, an angle between an axis of the inspection channel 201 and the conveyor belt 3 ranges from —15° to 15°.

According to some embodiments of the present disclosure, two sliding platforms 6 and two second driving devices 7 are provided. The two X-ray sources 202 are respectively disposed on the two sliding platforms 6, and the ray emission heights of the two X-ray sources 202 may be the same or different.

So far, the embodiments of the present disclosure have been described in detail with reference to the accompanying drawings. It should be noted that implementations not shown or described in the drawings or the specification are all forms known to those of ordinary skill in the art and are not elaborated here. In addition, definitions of the above components are not limited to the specific structures, shapes, or manners mentioned in the embodiments, and those of ordinary skill in the art may make simple changes or substitutions.

It should also be noted that in the specific embodiments of the present disclosure, unless otherwise stated to the contrary, the numerical parameters in the specification and the appended claims are approximate values that may be changed according to desired characteristics obtained through the contents of the present disclosure. Specifically, all numbers representing dimensions, ranges, conditions, etc., of compositions used in the specification and the claims should be understood as being modified by the term “about” in all cases. Generally speaking, it means that the specific number may vary by ±10% in some embodiments, ±5% in some embodiments, ±1% in some embodiments, and ±0.5% in some embodiments.

Those skilled in the art may understand that the features recorded in various embodiments and/or claims of the present disclosure may be combined and/or integrated in various ways, even if such combinations or integrations are not explicitly described in the present disclosure. In particular, without departing from the spirit and teachings of the present disclosure, the features recorded in the various embodiments and/or claims of the present disclosure may be combined and/or integrated in various ways. All such combinations and/or integrations fall within the scope of the present disclosure.

The specific embodiments described above provide further detailed explanations of the objectives, technical solutions, and beneficial effects of the present disclosure. It should be understood that the above are merely specific embodiments of the present disclosure and are not intended to limit the present disclosure. Any modifications, equivalent substitutions, improvements, etc., made within the spirit and principles of the present disclosure should be included in the scope of protection of the present disclosure.

1. An inspection device for a cavity-type object, comprising:
a base;
an inspection chamber rotatably disposed on the base, wherein the inspection chamber comprises:
an inspection chamber body, wherein an inspection channel with two open ends is formed inside the inspection chamber body;
an X-ray source disposed on a first side surface of the inspection channel; and
a radiation detector disposed on a second side surface of the inspection channel opposite to the first side surface;
a conveyor belt extending through the inspection channel between the X-ray source and the radiation detector, wherein the conveyor belt is configured to suspend and transport an object to be inspected; and
a first driving device disposed on the base, wherein the first driving device is configured to drive the inspection chamber to rotate relative to the base, so as to change an angle between rays emitted by the X-ray source and the conveyor belt,
wherein the conveyor belt is configured such that when the angle is equal to 90°, a distance between the conveyor belt and the first side surface is less than distance between the conveyor belt and the second side surface.

2. The inspection device according to claim 1, wherein two X-ray sources are provided, and the two X-ray sources are horizontally arranged side by side and spaced apart on the first side surface of the inspection channel.

3. The inspection device according to claim 2, further comprising:
an image generation device communicatively connected to the radiation detector, wherein the image generation device is configured to generate a three-dimensional image of the object to be inspected based on received ray signals.

4. The inspection device according to claim 3, wherein the object to be inspected is an animal carcass, and the three-dimensional image comprises at least one of a skeletal image of the animal carcass, a muscle distribution image of the animal carcass, and a lean-fat contrast image of the animal carcass.

5. The inspection device according to claim 2, wherein the inspection chamber further comprises a vertical movement assembly, and the vertical movement assembly comprises:
a guide rail disposed vertically on a side wall of the inspection channel;
a sliding platform slidably connected to the guide rail, wherein the two X-ray sources are disposed on the sliding platform; and
a second driving device configured to drive the sliding platform to move in a vertical direction so as to change a ray emission height.

6. The inspection device according to claim 1, wherein the first driving device comprises a servo motor.

7. The inspection device according to claim 5, wherein the second driving device comprises any one of a linear motor, a pneumatic cylinder, and a hydraulic cylinder.

8. The inspection device according to claim 5, wherein an apparatus installation chamber is provided inside the inspection chamber, two through holes extending vertically are provided on a side wall of the apparatus installation chamber to connect the apparatus installation chamber and the inspection channel, and the two X-ray sources are configured to emit rays to the radiation detector through the two through holes respectively.

9. The inspection device according to claim 1, wherein an angle between an axis of the inspection channel and the conveyor belt ranges from −15° to 15°.

10. The inspection device according to claim 5, wherein two sliding platforms and two second driving devices are provided, the two X-ray sources are respectively disposed on the two sliding platforms, and ray emission heights of the two X-ray sources 202 may be the same or different.
